SEQUENCE LISTING (15 sequences)

SEQ ID NO: 1 - protein, 7 aa, Homo sapiens
  misc_feature (4)..(4): Xaa can be any naturally occurring amino acid
DSFXIXE

SEQ ID NO: 2 - protein, 7 aa, Homo sapiens
DSFFIVE

SEQ ID NO: 3 - protein, 7 aa, Homo sapiens
DSFYIIE

SEQ ID NO: 4 - protein, 15 aa, Homo sapiens
  misc_feature (1)..(5): Xaa can be any naturally occurring amino acid
XXXXXDSFXIXEXXX

SEQ ID NO: 5 - protein, 15 aa, Homo sapiens
  misc_feature (1)..(5): Xaa can be any naturally occurring amino acid
XXXXXDSFFIVEXXX

SEQ ID NO: 6 - protein, 15 aa, Homo sapiens
  misc_feature (1)..(5): Xaa can be any naturally occurring amino acid
XXXXXDSFYIIEXXX

SEQ ID NO: 7 - protein, 15 aa, Homo sapiens
QGDSADSFFIVESGE

SEQ ID NO: 8 - protein, 15 aa, Homo sapiens
QGASADSFFIVESGE

SEQ ID NO: 9 - protein, 15 aa, Homo sapiens
QAASAASAAIVASGA

SEQ ID NO: 10 - protein, 15 aa, Homo sapiens
QAASAASFFIVASGA

SEQ ID NO: 11 - protein, 15 aa, Homo sapiens
QAASADSFFIVASGA

SEQ ID NO: 12 - protein, 15 aa, Homo sapiens
QAASADSFFIVASGE

SEQ ID NO: 13 - protein, 15 aa, Homo sapiens
QGDSADSFFIVASGE

SEQ ID NO: 14 - protein, 15 aa, Homo sapiens
QGASADSFFIVESGE

SEQ ID NO: 15 - protein, 15 aa, Homo sapiens
QGDSADSAAIVESGE

(19) United States
(12) Patent Application Publication
(10) Pub. No.: US 2015/0005237 A1
(43) Pub. Date: Jan. 1, 2015

(54) PEPTIDIC INHIBITOR OF SIGNAL TRANSMISSION FROM G-ALPHA-S TO G-ALPHA-I-COUPLED RECEPTOR CASCADES

(71) Applicant: UNIVERSITÄT INNSBRUCK, Innsbruck (AT)

(72) Inventors: Eduard Stefan, Mutters (AT); Verena Bachmann, Innsbruck (AT); Roland Huber, Innsbruck (AT)

(73) Assignees: UNIVERSITÄT INNSBRUCK, Innsbruck (AT); UNIVERSITÄT INNSBRUCK, Innsbruck (AT)

(21) Appl. No.: 14/366,874

(22) PCT Filed: Dec. 5, 2012
(86) PCT No.: PCT/EP2012/074489

§ 371 Date: Jun. 19, 2014

(30) Foreign Application Priority Data
Dec. 19, 2011 (EP) 11009942.1

(51) Int. Cl. C07K 7/08; G01N 33/68; G01N 33/543; A61K 38/10

(52) U.S. Cl. CPC C07K 7/08; A61K 38/10; G01N 33/6893; G01N 33/54306; C07K 2319/00; G01N 2500/04; G01N 2800/325; G01N 2333/4719
USPC 514/9.7; 530/326; 514/19.3; 514/16.4; 514/21.5; 436/86; 506/9

(57) ABSTRACT

The present invention relates to novel peptidic inhibitors of signal transmission from Gαs- to Gαi-coupled receptor cascades and methods for their use.

The present invention relates to novel peptides and their use in screening, preparation, diagnostic and therapeutic methods.

Living cells have evolved regulatory systems to distinguish and integrate extracellular signalling cues. Signals received on the cell surface by receptors are transmitted, processed and amplified via intracellular effectors of well-tuned interconnected signalling cascades or circuits.

Heterotrimeric G proteins (Guaninnucleotide-binding protein, GTP-binding protein) are membrane-located signal transducers that communicate signals from many hormones (e.g. glucagon, epinephrine), neurotransmitters (e.g. dopamine, acetylcholine), chemokines (IL-8) and local mediators (LPA) via activation of G protein coupled receptors (GPCRs). The GPCR family has enormous pharmacological importance, as demonstrated by the fact that more than 30% of approved drugs elicit their therapeutic effect by selectively acting on distinct known members of this family. Analysis of the human genome reveals more than 800 putative GPCRs including a considerable number with unknown physiological function or ligands. Hence, identification, classification and characterization of GPCR cascades and their connections and crosstalk remain a major focus of molecular pharmacology.

Once a ligand activates a specific GPCR through binding signaling occurs basically via four main G protein families (Gαs, Gαi, Gαq/11 and Gα12/13) to regulate such cellular machinery as metabolic enzymes, ion channels and transcriptional regulators impacting on e.g. cardiac function, glucose metabolism, synaptic plasticity, proliferation, chemotaxis or pacemaker activity. In the canonical description of GPCR signalling, Gαs-coupled receptors activate adenylate cyclase (AC) mediated synthesis of second messenger Adenosine 3′-5′-Cyclic Monophosphate (cAMP), which in turn binds to regulatory subunits of PKA (Reg, hereinafter called R, which occur as isoforms RIα, RIβ, RIIα and RIIβ), inducing conformation changes that result in dissociation of tetrameric R₂:PKAc₂ (Cat=catalytic PKA subunits, hereinafter called PKAc) complexes, resulting in active PKAc subunits. Gαi-coupled receptors activate MAP kinase (MAPK) signalling cascades and simultaneously inhibit AC activities. To date, regulatory PKA subunits have been thought of as passive inhibitors of catalytic PKA activity. Thus, the second messenger (cAMP)-dependent protein kinase A (PKA) is a canonical effector of signal transduction in response to hormones that bind to many cell surface receptors including ‘G-protein’-coupled receptors (GPCR). Classically, hormone-mediated activation of GPCRs coupled either to G-protein families Gαs or Gαi stimulate or inhibit cAMP-generating adenylate cyclases.

However, up to date the function and networking of the different hormone-dependent pathways is not fully understood. Since a full understanding of said hormone-related cascades is a very valuable tool to understand hormone-related diseases and their impact on parallel signalling cascades, there is a need to provide such tools for more thoroughly analysing pharmacologically relevant interactions between the hormone-related signal cascades and to provide tools to control and manipulate these cascades with the aim to provide useful therapeutic means to prevent or treat diseases relating to said signal cascades.

Thus, the present invention is based on the technical problem of roviding tools for screening for pharmaceutically active and target-orientated substances which affect GPCR signalling. Such substances allow to treat G protein-related diseases and provide strategies to screen for further target-related bioactive compounds.

The present invention solves its underlying problem by providing a PKA RII-derived peptide, in particular a PKA RIIα or PKA RIIβ derived peptide, hereinafter also called Reggai peptide. Said peptide is derived from a part of the regulatory subunit type II, preferably IIα or IIβ of the cAMP-dependent protein kinase A (PKA) and according to the present invention represents the sole binding site of the PKA RII subunits for Gαi. Preferably, the Reggai peptide is the RIIβ-derived peptide.

The present Reggai peptide in a particularly preferred embodiment is able to prevent the binding of PKA RII, preferably RIIα and RIIβ (PKA regulatory subunit IIα and IIβ) to Gαi, in particular receptor-activated Gαi.

In a preferred embodiment, the Reggai peptide prevents the cAMP-RIIα and cAMP-RIIβ-mediated enhancement of hormone/agonist-triggered Gαi signalling.

Thus, the present invention provides the teaching on a novel and evolutionary conserved mechanism of GPCR synergism between two of the best studied families of Gα proteins (Gαi and Gαs) through the formation of a dynamic protein-protein interaction between the regulatory subunit of the prototypical protein kinase A (PKA; part of the Gαs cascade) and the Gαi protein. The present invention discovers that cAMP-activated regulatory subunits of the protein kinase A (PKA) specifically bind to the cAMP antagonizing G-protein-αi. This dynamic binary interaction augments the sensitivity, amplitude and duration of trimeric G-protein Gαi:βγ activity to stimulation of G-protein coupled receptors but does not activate Gαi signaling alone. The mechanism provides a unique example of a critical evolutionary adaptation that provides for a simple and high-fidelity modulation of sensory response to various extracellular hormon signals. It is a mechanistically highly conserved mechanism that is conserved from yeast to man for decisions of cell fate, such as proliferation and differentiation states and possibly for a myriad of cellular and whole organism adaptations, from control of blood pressure to chemotaxis and also to consolidation of memory.

Thus, the present invention is in one of its aspects directed to the identification and provision of the PKA RII-derived peptide, in particular the PKA RIIα or PKA RIIβ-derived peptide, preferably the PKA RIIβ-derived peptide, hereinafter also called ‘Reggai’ peptide, which specifically blocks signal transmission from Gαs- to Gαi-coupled receptor cascades. The Reggai peptide specifically prevents sensitization and enhancement of ligand triggered Gαi-signaling responses without having a direct effect on PKA activities or directly on Gαi (without a bound Gαi-PCR ligand) alone.

Similar signal integration and tuning of Gαs- and Gαi-coupled hormone responses is likely common to all cell types of eukaryotes and this under physiological as well as pathological states. Components of Gαs- and Gαi-coupled receptor pathways are prominent targets for therapeutic intervention in a number of human diseases. Thus, consequences of PKA mediated tuning of Gαi activities on drug actions will need to be considered when evaluating their therapeutic and off-target effects. The present invention opens a potential avenue for reducing Gαi signaling through MAPK pathways (the key kinase involved in proliferation) and is of potential utility in cancer therapies. Pharmacological targeting of cAMP-bound PKA regulatory subunits via direct competition for the interaction with Gαi with candidate drugs may prove valuable therapeutic strategies especially in the prospect of polypharmacology.

In a preferred embodiment, the present Reggai peptide is provided in essentially pure, preferably pure form, preferably the present peptide is provided in isolated form.

In a preferred embodiment of the present invention a PKA RII-derived peptide (Reggai peptide) is provided consisting of 12 to 17, preferably 12 to 15, most preferably 15 amino acids, wherein said peptide has a core element of at least seven amino acids being DSFXIXE (SEQ ID No. 1), wherein X is any amino acid, in particular any naturally occurring amino acid, in particular any naturally occurring amino acid in humans.

In a preferred embodiment of the present invention the PKA RII-derived peptide (Reggai peptide) is a PKA RIIα-derived peptide consisting of 12 to 17, preferably 12 to 15, most preferably 15 amino acids, wherein said peptide has a core element of at least seven amino acids being DSFYIIE (SEQ ID No. 3).

In a preferred embodiment of the present invention the PKA RII-derived peptide (Reggai peptide) is a PKA RIIβ-derived peptide (Reggai peptide) consisting of 12 to 17, preferably 12 to 15, most preferably 15, amino acids, wherein said peptide has a core element of at least seven amino acids being DSFFIVE (SEQ ID No. 2).

In a preferred embodiment the other amino acids of the peptide, that means the remaining non-specified amino acids adding up to the overall amount of 12 to 17, preferably 12 to 15, amino acids, that means five to ten, preferably five to eight, amino acids, can be any amino acid, in particular any naturally occurring amino acid, in particular any naturally in human beings occurring amino acid.

In a furthermore preferred embodiment, the PKA RII-derived peptide, preferably the PKA RIIα- and PKA RIIβ-derived peptide, having the above-identified amino acid sequence of the present invention consists of 15 amino acids.

Thus, the present peptides are characterised by a limited length of 12 to 17, in particular 12 to 15, preferably 15, amino acids and being characterised by the necessity of having at least the amino acid sequence of SEQ ID NO. 1, or of SEQ ID No. 2 or of SEQ ID No. 3 as its core element.

In a furthermore preferred embodiment of the present invention the peptide is selected from the group consisting of XXXXXDSFXIXEXXX (SEQ ID No. 4), XXXXXDSFFIVEXXX (SEQ ID No. 5) and XXXXXDSFYIIEXXX (SEQ ID No. 6), wherein X is any amino acid, in particular wherein X is any naturally occurring amino acid, in particular wherein X is preferably any naturally in human beings occurring amino acid. Particularly preferred is the peptide with SEQ ID No. 5.

In a furthermore preferred embodiment of the present invention the PKA RIIβ-derived peptide is QGDSADSFFIVESGE (SEQ ID No. 7) or QGASADSFFIVESGE (SEQ ID No. 8).

Thus, the present teaching provides in a preferred embodiment a PKA RIIβ-derived peptide of SEQ ID No. 7 in isolated form, which in its naturally occurring non-isolated context forms an integral part of the naturally occurring PKA RIIβ.

The present invention furthermore provides functional variants of SEQ ID No. 7. Functional variants are characterised by having the same binding property, that means having the ability to bind to Gαi, and having the same length, that means having a length of 12 to 17, preferably 12 to 15, in particular 15, amino acids. Such a functional variant is characterised for instance by SEQ ID No. 8 or is characterised by any amino acid sequence comprising the core element DSFXIXE (SEQ ID No. 1), DSFFIVE (SEQ ID No. 2) or DSFYIIE (SEQ ID No. 3) and having the length specified herein.

In the context of the present invention, a PKA RII-derived peptide, preferably a PKA RIIα- and PKA RIIβ-derived peptide, mentioned herein is a peptide in isolated and purified form, for intance being synthesised or being recombinantly produced.

In a preferred embodiment, the present invention provides nucleotide molecules comprising nucleotide sequences, in particular DNA sequences, encoding the present peptides. In a furthermore preferred embodiment, the present invention provides vectors containing said nucleotide sequences, host cells comprising said vectors and methods to use said nucleotide sequences and/or host cells, to produce the peptides of the present invention. In a preferred embodiment of the present invention there is provided a polyclonal or monoclonal antibody specifically directed, that means specifically recognising, the Reggai peptide of the present invention.

In a particularly preferred embodiment of the present invention, the peptide of the present invention is a fusion peptide, that means is fused to another peptide or protein.

In a furthermore preferred embodiment of the present invention the peptide or the fusion peptide of the present invention is labelled. In a particularly preferred embodiment of the present invention the peptide or fusion peptide is labelled with a radioactive label, a photo label, a fluorescence label, a chemotactic label, a toxic label or an immuno label.

In the context of the present invention, a G protein is a GTP-binding protein, in particular a heterotrimeric G-protein, in particular a membrane-located heterotrimeric G-protein.

In the context of the present invention, RII is preferably RIIα or RIIβ, most preferably RIIβ.

In a preferred embodiment, Gαi is at least one of the group consisting of Gαi1, Gαi2 and Gαi3.

In the context of the present invention, the Gαi protein is preferably the Gαi3 protein.

In the context of the present invention the term “cAMP-bound PKA RII” refers to PKA RII which is dissociated from the PKA holoenzyme and is bound to cAMP. Preferably, the cAMP-bound PKA RII is the cAMP-bound PKA RIIα or RIIβ subunit, most preferably the cAMP-bound RIIβ subunit.

In a furthermore preferred embodiment of the present invention, the present Reggai peptide is for use in a prophylactic or therapeutic method for treating a subject in need thereof, in particular a mammal, preferably human. In a preferred embodiment of the present invention the treatment is a treatment for a G protein-related disease, in particular a trimeric G protein-related disease.

In a preferred embodiment of the present invention, a trimeric G protein-related disease is a disease whose etiology, cause or treatment is significantly being related to hormone-binding, neural transmitter binding, chemokine binding or mediator binding to G protein-coupled receptors.

In a particularly preferred embodiment the, preferably trimeric, G protein-related disease is cancer or a hormone-related disease. In fact, distinct mechanisms have been proposed to explain how cAMP regulate cell type specific MAPK signalling which modulates cell growth and proliferation either positively or negatively. In fact, human endocrine tumours have mitogenic MAPK activities caused by activating mutations of Gαi too. Since the present invention shows that the application of the present Reggai peptide reduces cAMP-RII-mediated activation of MAPK via Gαi, the present peptides are suitable to reduce Gαi activities and therefore tumor proliferation.

A further, preferably trimeric, G protein-related disease is heart failure. In a particular embodiment of the present invention, the present Reggai peptide is used to treat heart failure. Said treatment is based on the teaching that one characteristic of the heart failure is upregulation of Gαi2 at the protein and transcript level. It is known that Gαi signalling contributes to the development of dilated cardiomyopathy. Therefore, application of the present Reggai peptide reducing Gαi signalling is suitable to prevent or treat heart failure.

In a preferred embodiment the present Reggai peptide is used together with a pharmaceutically acceptable carrier.

In a furthermore preferred embodiment of the present invention, the present Reggai peptide is used in an amount sufficient to treat the G protein-related disease.

Thus, the present invention also relates to methods of treating a subject, in particular a human or animal subject, by administering the present Reggai peptides or pharmaceutical compositions comprising said Reggai peptides in an amount sufficient to treat the subject in need thereof.

Furthermore preferred is the use of the present Reggai peptide together with at least one further pharmaceutically active substance which latter is also of use in treating a, preferably trimeric, G protein-related disease. Thus, the present invention provides a pharmaceutical composition comprising at least two pharmaceutically active substance, one of them being the present Reggai peptide.

In particular, this teaching is based on the present showing that Gαi signalling sensitivity is increased by Gαs-coupled receptor signalling. This means that the sensitivity of Gαi-coupled receptor signalling processes can be increased by pharmaceutically active substance that positively act on Gαs-coupled receptors. The present Reggai peptide therefore preventing this cross talk results in a reduction of undesired side effects resulting from enhancing Gαi-mediated signalling. In a preferred embodiment of the present invention, the further pharmaceutically active substance optionally being used together with the present Reggai peptides is a dopamine D2 receptor, is the β-opioid receptor or the 5-HT_{1A} receptor.

Dopamine receptors are implicated in neurological processes for instance memory and learning. Abnormal dopamine receptor signalling and dopamine organic nerve function is implicated in neuropsychiatric disorders.

μ-opioid receptors are involved in drug addiction.

The 5-HT_{1A} receptor binds the endogenous neurotransmitter serotonin. The agonist decreases blood pressure and heart rate.

Furthermore, the present invention provides for modification of the efficacy of Gαi-coupled receptor agonists, antagonists or both by treatment with Gαs-coupled receptor drugs, preferably by using the present Reggai peptide to prevent this interaction.

The present invention also provides a method for screening candidate drugs, which method comprises a) providing a peptide structure comprising, in particular consisting of, a Reggai peptide according to the present invention and at least one candidate drug, b) bringing the compounds of step a) in physical contact with each other under binding conditions and c) analysing the binding of the at least one candidate drug to the peptide structure.

The present invention also relates to the method according to the above, wherein in step a) a cAMP-bound PKA RII, a G protein αi or both are provided and brought into physical contact with the other compounds in step b).

The present invention also relates to a method for screening candidate drugs, which method comprises a) providing a cAMP-bound PKA RII, a G protein αi (Gαi) and at least one candidate drug, b) bringing the compounds of step a) into physical contact with each other under binding conditions and c) analysing the effect of the candidate drug on the binding of the at least one cAMP-bound PKA RII to Gαi.

The present invention also relates to a method of preparing a cAMP-bound PKA RII comprising a) providing a source containing cAMP-bound PKA RII and a source containing the G protein αi, b) contacting the source containing PKA RII with the source containing the G protein αi under conditions allowing for the binding of PKA RII to Gαi, c) obtaining the bound complex of PKA RII/Gαi and d) separating the PKA RII from Gαi.

The present invention also relates to a method of preparing G protein αi comprising a) providing a source containing cAMP-bound PKA RII or the present Reggai peptides and a source containing the G protein αi, b) contacting the source containing PKA RII or the peptides with the source containing G protein αi under conditions allowing for the binding of PKA RII or the present Reggai peptides to Gαi, c) obtaining the bound complex of PKA RII/Gαi or the present peptide/Gαi and d) separating Gαi from PKA RII or the present Reggai peptides.

Preferably, in the context of the present invention, in particular in the methods of the present invention, PKA RII is PKA RIIα or PKA RIIβ, most preferred PKA RIIβ.

In a preferred embodiment of the present invention, the present methods are in vitro methods.

In a furthermore preferred embodiment of the present invention “bringing the compounds into physical contact with each other under binding conditions” or “contacting under conditions allowing for the binding” is meant to refer to a step of mixing and incubating the compounds in question and providing conditions under which the compounds are potentially able to bind to each other in case they have the required binding capability and no interfering substance, for instance a particular competitive candidate drug, is present.

In a preferred embodiment, the step of “contacting” the compounds or “bringing into physical contact” is done in an aqueous medium.

In a particularly preferred embodiment of the present invention, analysing the effect of the candidate drug involves a comparison of the binding of the at least one cAMP-bound PKA RII to Gαi in the presence and absence of the candidate drug.

In the context of the present invention, a candidate drug is a substance which is to be analysed in term of its binding behaviour to a compound in question, in particular with the aim to gain more information on the candidate drug and/or the compounds to which it is supposed to bind, preferably with the aim to provide pharmaceutically active substances, preferably to identify the candidate drug as a pharmaceutically active substance.

The present inventors identified and successfully applied the Reggai peptide directly in the living cells, in particular cell culture, HEK293 cell line, and changed the original and conserved Reggai sequence in a model organism (budding yeast) to prevent signal transmission from Gαs- to Gαi-cascades. The Reggai peptide had no effect on sole activation of either cascade, just in the case of simultaneous activation of both types of receptors the Gαs triggered and PKA-mediated elevation of Gαi activities was abolished. In the mammalian system the present inventors used biochemical tools to measure changes of cAMP levels, G protein activities and MAPK activation downstream of Gαi-coupled receptors upon formation of the dynamic interaction between PKA and Gαi.

The present function of cAMP RII-bound Gαi reported is to be understood in the context of known mechanisms of GPCR signalling dynamics. First, adenylyl cyclases are regulated, positively by Gαs and negatively by Gαi. Further, receptor-activated kinases (including PKA) regulate adenylyl cyclases by phosphorylation. The present mechanism elaborates on these feed-forward and feedback mechanisms at the level of adenylate cyclase. In addition, GPCRs themselves are targets of receptor-activated kinases leading to circuits of desensitisation or switching of the primary G protein coupling. The present cAMP-RII mediated Gαi modulation is, however, part of a general process of G protein switching and sequential activation of Gαs and Gαi cascades, which has previously been proposed for human β₂-adrenergic receptors. Second, the cAMP-dependent RII:Gαi interaction creates a feedback loop, whose topology suggests the potential of participating in oscillating cycles of cAMP synthesis. The consequences of this feedback loop to existing models for oscillating cAMP signalling will need to be accessed.

Third, varying cellular expression ratios of R subunits and Gαi, may affect Gαi-coupled receptor signalling in different cell types or conditions. Fourth, PKA R subunits are anchored to an assortment of AKAP scaffolding proteins, which tether and organize signalling complexes at specific sites in the cell. Our results thus imply that cAMP-bound RII brings these scaffolds into proximity of Gαi and its receptor/effectors which could serve to further organise compartmentalised signalling complexes and redirect the flow of information between GPCR cascades. Fifth, we present evidence that cAMP-bound RII increases the ligand sensitivity of GPCRs, which could affect the potency of ligands or drugs, designed to act on specific Gαi coupled receptors. Distinct mechanisms have been proposed to explain how cAMP regulates cell type specific MAPK signalling either positively or negatively. For instance, human endocrine tumors have mitogenic MAPK activities caused by activating mutations of Gαi. The cAMP-triggered RII modulation of Gαi-coupled receptor signalling may be a general mechanism through which Gαs-coupled receptors modulate MAPK pathways under normal or pathological conditions. Sixth, since both Gαi and Gαs coupled receptors and downstream effectors are among the most common targets for therapeutic intervention in a number of diseases, cAMP-RII modulation of Gαi signalling may have unintended consequences to normal physiology that will need to be accounted for in the development of drugs. For instance, one avenue for creating PKA inhibitors is based on cAMP analogues that compete with cAMP for R subunit binding, but do not cause R:PKAc dissociation and therefore prevent PKAc activation. By preventing dissociation of the holoenzyme complex, these molecules may have unforeseen consequences due to their prevention of regulatory PKA subunit binding to effectors, including but perhaps not limited to the Gαi proteins.

Finally, the present teaching also opens a potential avenue for reducing Gαi signalling through MAPK pathways which is of potential utility in cancer therapies. Pharmacological targeting of cAMP-bound R subunits or direct competition for RII:Gαi interactions for example with peptide variants based on the present Reggai peptide is a prove valuable therapeutic tool.

Thus, the present invention provides the PKA RII, in particular RIIα or RIIβ, preferably the RIIβ-derived peptide, i.e. the Reggai peptide, which specifically inhibits the formation of a signalling crosstalk between two prevalent and critically regulated GPCR cascades (Gαs and Gαi-coupled receptors), without affecting their signal transmission in case just one cascade is activated. The peptide provided can be used to reduce off-target effects of drugs acting on Gαs- or Gαi cascades by preventing the direct physical interaction with and further activation of the G protein Gαi, thereby potentially increasing specificity of agonists/antagonists and being of potential importance in polypharmacology approaches.

The present invention in preferred embodiments provides the following:

1. The teaching that components of Gαs-coupled receptor cascades directly modulate Gαi-mediated signaling, which can be inhibited by applying the Reggai peptide.
2. The present identified interaction and function is evolutionary conserved and can be found in the baker's yeast as well, which underlines its ubiquitous occurrence, which is relevant for the whole palette of pharmacological important Gαi- or Gαs-coupled receptors.
3. One advantage of the present Reggai peptide is that it solely affects signal transmission once both Gαi or Gαs cascades are activated. The present Reggai peptides has no effect on Gαs- or Gαi-casades, respectively, in case only one of them is activated at a given time.
4. The Reggai peptide represents the sole and highly conserved interaction site necessary for interaction and modulation of ligand triggered Gαi activities.
5. The Reggai peptide is useful to be applied in combination with drugs acting either on components of Gαs- or Gαi-pathways (polypharmacology). The present Reggai peptide is suitable to prevent off-target effects related to the described mechanism of PKA mediated changes of Gαi activities impacting on cAMP levels and MAPK activities (link to proliferation).
6. The present teaching that Gαi signaling sensitivity is increased by Gαs-coupled receptor signaling means that the sensitivity of Gαi-coupled receptor signaling processes could be increased by drugs that positively act on Gαs-coupled receptors.
7. The efficacy of Gαi-coupled receptor agonists and/or antagonists could be modified by treatment with Gαs-coupled receptor drugs and signalling crosstalk could be prevented by the present Reggai peptide. Currently, such agonists and antagonists are used for treating diseases of aberrant cell proliferation such as cancers, e.g. compounds acting on CXCR4 and Smoothened receptors (both are putative Gαi-coupled receptors) and e.g. these can be used optionally together with the present peptides.

Further preferred embodiments are the subject matter of the subclaims.

The sequence listing shows:

SEQ ID No. 1 shows the core sequence of seven amino acids of the present Reggai sequence, in particular the PKA RII-derived sequence, wherein X is any amino acid,

SEQ ID No. 2 shows a core sequence of seven amino acids of the present Reggai sequence, in particular the PKA RIIβ-derived sequence,

SEQ ID No. 3 shows a core sequence of seven amino acids of the present Reggai sequence, in particular the PKA RIIα-derived sequence,

SEQ ID No. 4, 5 and 6 show the general amino acid sequences of the present Reggai sequences comprising the core sequence, also called core element, of SEQ ID No. 1 (SEQ ID No. 4), of SEQ ID No. 2 (SEQ ID No. 5) and of SEQ ID No. 3 (SEQ ID No. 6), and consisting of 15 amino acids, wherein a X (or Xaa) is any amino acid, in particular any naturally occurring amino acid, preferably in a human,

SEQ ID No. 7 represents the PKA RIIβ-derived peptide sequence of the present invention and being part of the naturally occurring PKA RIIβ,

SEQ ID No. 8 represents a functional variant of the Reggai peptide of SEQ ID No. 7,

SEQ ID No. 9 to 15 represent non-inventive comparative peptide sequences not having the core sequence of SEQ ID No. 1.

In the following the invention is described by way of non-limiting examples and the accompanying figures.

The figures show:

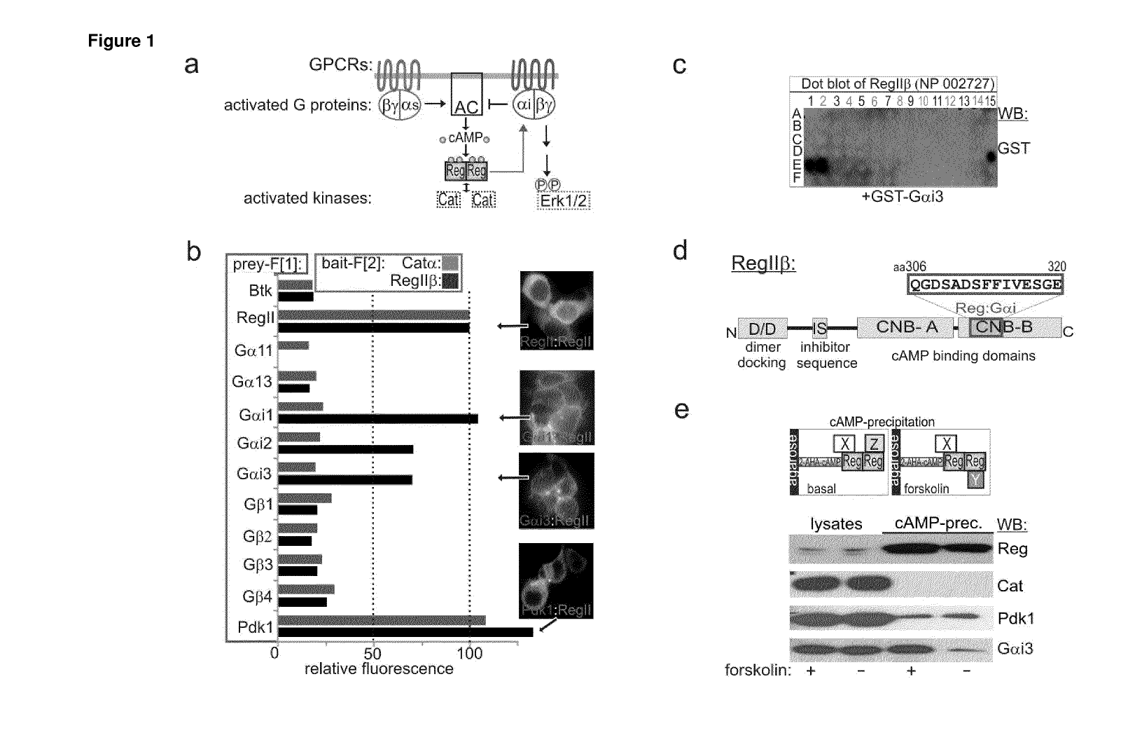

FIG. 1 The regulatory PKA subunit forms cAMP-dependent complexes with Gαi isoforms. a, Schematic representation of GPCR cascades linked to cAMP turnover. Gα-proteins modulate adenylyl cyclase (AC) activities. Activation is shown by arrows, inhibition by T-bars. Right-angled arrow indicates novel feedback of Reg to G protein αi. Dotted lines indicate activated kinases (subunits of PKA: R or Reg, PKAc or Cat; P stands for phosphorylated MAPK). b, Left panel: Fluorometric analysis of transiently transfected COS7 cells in suspension co-expressing PKA subunits as bait tagged with Venus YFP PCA fragment [2] (RegIIβ or RIIβ-F[1], Catα-F[2] or PKAc-α-F[2]) with the indicated prey proteins fused to F[1]. Right panel: Fluorescence images of HEK293 cells co-expressing indicated protein pairs. c, Spotted peptides (25 mers, 20 aa [amino acid] overlap) of RIIβ (accession number: NP_{—}002727, aa1-25 spotted on A1, aa5-30 on A2 etc.) were overlaid with recombinant GST-Gαi3. d, Modular structure of RIIβ with indicated binding interface for R:Gαi; (box). e, cAMP precipitation strategy: Resin coupled cAMP analogue 2AHA-cAMP binds endogenous R/Reg subunits; overexpressed prey proteins (X, Y or Z) co-precipitate. cAMP precipitation of endogenous PKA subunits from COS7 cells transiently expressing Pdk1 or Gαi3; ±15 min forskolin (100 μM).

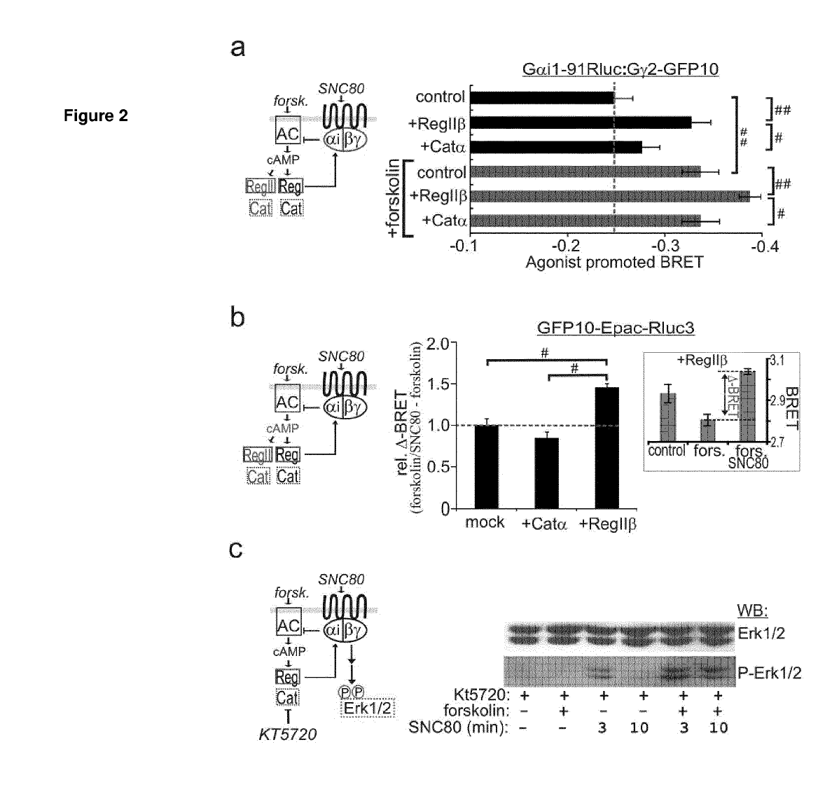

FIG. 2 cAMP-bound R/Reg subunit enhances ligand triggered Gαi activation, AC inhibition and MAP kinase phosphorylation. a, Conformational changes of trimeric G-proteins measured in δOR-HEK293 cells coexpressing indicated BRET protein reporter pairs and PKA subunits following stimulation with forskolin (100 μM, 15 minutes) and then by SNC80 (1 μM, 3 minutes; ±SEM, n=3 independent experiments). b, cAMP levels measured in δOR-HEK293 cells coexpressing the cAMP sensing EPAC BRET reporter protein pairs and PKA subunits following stimulation with forskolin (50 μM, 12 minutes) and then SNC80 (1 μM, 6 minutes; ±SEM, representative experiment, n=3). Decrease in BRET ratio (Δ-BRET) indicates an increase in cAMP concentration in vivo. Inset shows forskolin (±SNC80)-induced cAMP change measured with EPAC-BRET in cells overexpressing RegIIβ (±SEM). c, Effects of combinations of forskolin (100 μM, 15 minutes) and SNC80 (1 μM, minutes) on Erk1/2 phosphorylation in δOR-HEK293 cells; 60 minutes pre-treatment with KT5720 (2 μM), representative experiment of n=3. Statistical significance was assessed using a paired student's t-test (#, p-values <0.05; ##, p-values <0.01). Treatments (italic) and readouts are indicated in the schematics.

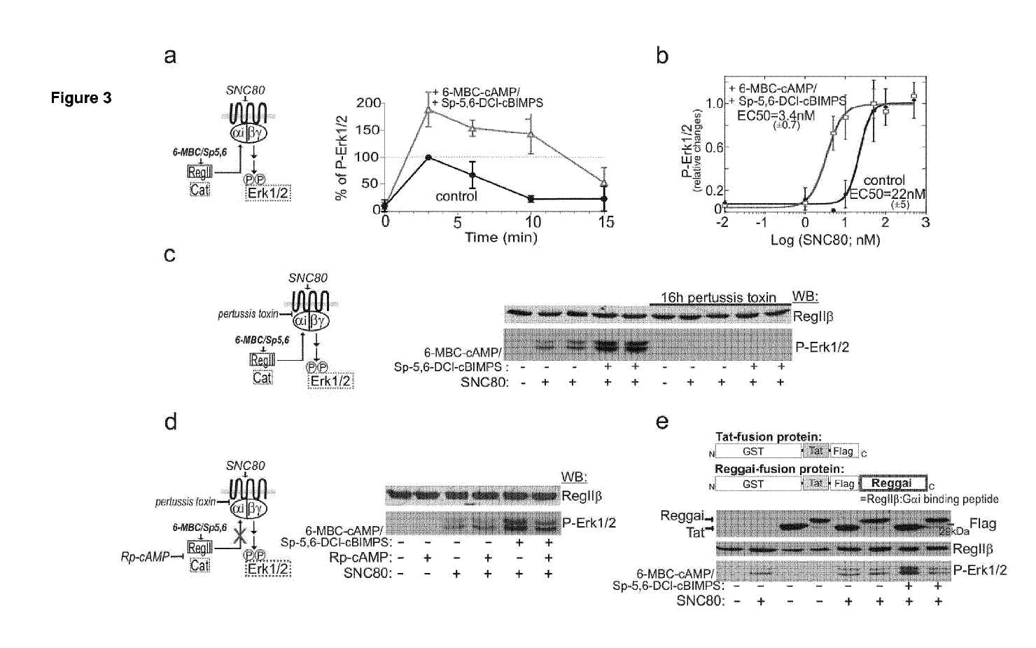

FIG. 3 cAMP activated RII:Gαi enhances MAP kinase pathway sensitivity and duration to δOR activation. a-e, Effects of indicated treatments (chemical modulators, overexpression, hybrid fusion proteins) and combinations of 6-MBC-cAMP, Sp-5,6-DCI-cBIMPS (each 50 μM, 15 minutes) and the selective δOR agonist SNC80 (1 μM) on Erk1/2 phosphorylation (P-Erk1/2) in δOR-HEK293 cells. RegIIβ serves as loading control. a, Time dependent effects of combinations of SNC80 (minutes) and 6-MBC-cAMP, Sp-5,6-DCI-cBIMPS on P-Erk1/2. b, Dose dependent effects of combinations of SNC80 (3 minutes) and 6-MBC-cAMP, Sp-5,6-DCI-cBIMPS on P-Erk1/2. c, Uncoupling of Gαi from the receptor through pre-treatment with pertussis toxin (16 hours, 100 ng/ml; representative experiment). d, Effect of pre-treatment with Rp-cAMP (250 μM; 60 minutes) on P-Erk1/2. e, Domain structure for the Reggai peptide fused to GST-TAT-Flag peptide. Treatments of δOR-HEK293 cells with either TAT control or the Reggai fusion peptides (each ˜3 μM; 30 minutes) on P-Erk1/2 are shown. a, b, d, e, Pre-treatment with KT5720 (60 minutes, 2 μM); c-e, representative experiment of at least n=3.

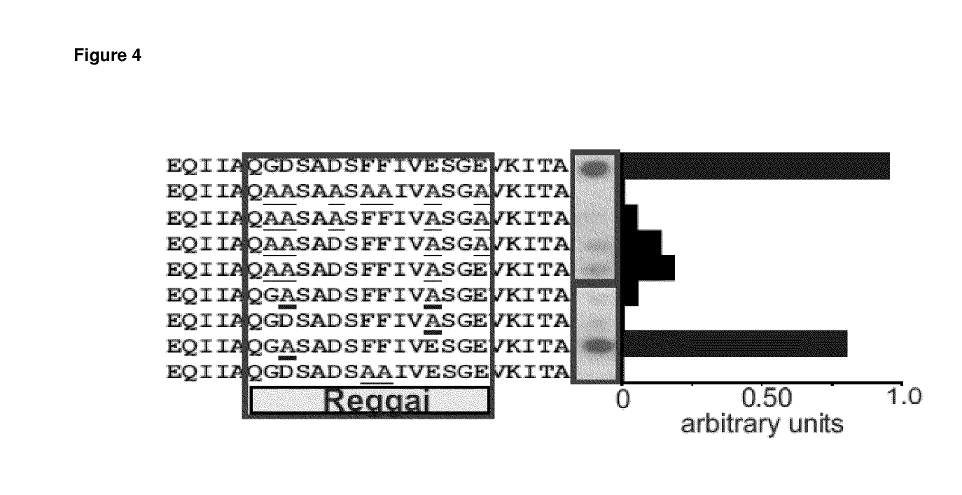

FIG. 4 Dot blot analysis of conserved amino acids (core element of SEQ ID No. 1, 2, extended sequences SEQ ID No. 4 and 5) in the Reggai sequence. Spotted peptides containing the Reggai sequence (25 mers) of RIIβ (Reg) (SEQ ID No. 7), its mutant functional variant (SEQ ID No. 8) and non-functional peptides not containing the Reggai core element of SEQ ID No. 1 variant (SEQ ID No. 9 to 15) (exchanged amino acids are underlined) were overlaid with recombinant GST-Gαi3. The box indicates the Reggai sequence. Densitometric analysis of the average of two independent dot blot experiments is shown as bar graph. FIG. 4 shows a sequence comparison of RIIβ PKA peptides of the present invention, namely SEQ ID No. 7 and 8 (SEQ ID No. 7 being the top sequence, while SEQ ID No. 8 being the 8^{th} sequence from the top) in comparison to peptides comprising amino acid sequences not comprising the core element of the present invention, namely that of any one of SEQ ID No. 1 to 3 (extended sequences SEQ ID No. 4 to 6). SEQ ID No. 9 to 15 in FIG. 4 (2^{nd} to 7^{th} and 9^{th} sequence) are non-inventive comparative peptides.

### EXAMPLES

### Materials and Methods

### a) Antibodies.

Primary antibodies used were monoclonal anti-RIIβ (BD Bioscience, #610626) anti-RIIα (BD Bioscience, #612242) and anti-PKAcα (BD Bioscience, #610981), rabbit anti-Gαi3 (Santa Cruz, #sc-262), monoclonal anti-active Gαi antibody (New East Biosciences, #26901), rabbit anti-Erk1/2 (Cell Signaling, #9102) and rabbit anti-phospho-Erk1/2 (Cell Signaling, #9101), monoclonal anti-CREB (Cell Signaling, #9104) and rabbit anti-phospho-Serine-CREB (Cell Signaling, #9198), monoclonal anti-GFP to detect F[2] of the Venus YFP PCA (Roche, #11814460001), monoclonal anti-Flag (Sigma, #F3165) and anti-GST (Sigma, #G1160).

### b) Construction of Plasmids.

Venus YFP PCA expression vectors and fusion constructs: The N-terminal fragment (1-158 aa [amino acids]; F[1]) and the C-terminal fragment (159-239 aa; F[2]) of the yellow fluorescent protein (YFP) variant ‘Venus’ were amplified by polymerase chain reaction (PCR), see MacDonald et al., Nat. Chem. Biol. 2, 329-337 (2006), Michnick et al., Nat. Rev. Drug Discov. 6, 569-582 (2007), Nagai et al., Nat. Biotechnol. 20, 87-90 (2002), Nyfeler et al., J. Cell Biol. 180, 705-712 (2008), Stefan et al., Proc. Natl. Acad. Sci. U.S.A. 104, 16916-16921 (2007).

The protein-fragment complementation assay (PCA) fragments were cloned into the multiple cloning site (MCS) of the eukaryotic expression vector pcDNA3.1 (Invitrogen). The PKA regulatory subunit (rat, type IIβ, RIIβ) and the PKA catalytic subunit (mouse, type α, PKAc) were amplified by PCR and subcloned into the 5′ to the Venus YFP PCA fragments referred to here as F[1] (N-terminal) or F[2] (C-terminal) complementary fragments. In the latter case, an oligonucleotide encoding a 10-amino-acid linker sequences (Gly-Gly-Gly-Gly-Ser)₂ was inserted between the cDNA and the PCA fragments. To screen for RIIβ and PKAc interacting proteins, we generated plasmids designed to serve as expression and destination vectors for lambda recombinase subcloning of mouse cDNAs harbored in Gateway™—ready entry vectors and upstream of Venus YFP PCA fragments (F[1], F[2]) for detection of protein:protein interactions. Venus YFP PCA fragments were subcloned into a pcDNA 3.1 expression vector with the appropriate Gateway™ reading frame cassette (Invitrogen) to generate the C- and N-terminal destination vectors for each PCA fragment. cDNAs of interest were subcloned into these destination vectors with LR (lambda recombinase) Clonase™ as recommended by the manufacturer (Invitrogen Corporation). Positive clones were identified by PCR amplification of a fragment of expected size using primers of common flanking sequence. GST fusion protein constructs: A fusion protein was constructed consisting of full-length Gαi3 and gluthatione S-transferase (GST) to be used in GST pulldown experiments and dot blot analysis. The cDNA for Gαi3 was amplified from rat heart Marathon-Ready cDNA (BD Biosciences) by PCR and cloned into the MCS of the GST expression vector pGEX-5X-1 via EcoRI/XhoI. Further a GST-RIIβ construct was provided. Oligos coding for the 15 amino acids of the binding interface of RIIβ:Gαi3 (‘Reggai peptide’) (SEQ ID No. 7) were cloned into the MCS of the GST expression vector pGEX-4T-2 (via EcoRI/XhoI) flanked upstream with the sequences for GST, Tat and Flag anti-genic peptide tag (see Becker-Hapak et al., Methods 24, 247-256 (2001); Pontier et al., Embo J. 25, 2698-2709 (2006)).

All GST fusion proteins were expressed in Escherichia coli (strain BL21). Induction, cell lysis and affinity purification of fusion proteins were performed as recommended by the supplier of the pGEX vectors (GE Healthcare).

### d) Fluorometric Analysis of Venus-PCA Tagged Proteins (Mammalian Cells).

COS7 cells grown in 12-well plates (in Dulbecco's Modified Eagle's Medium [DMEM, Invitrogen] supplemented with 10% fetal bovine serum[FBS]) were co-transfected with the Venus YFP PCA expression vectors (pcDNA3.1) coding for prey-F[1] or bait-F[2] using FuGENE6 reagent (Roche). 48 hours after transfection, cells were resuspended in phosphate buffered saline (PBS: 137 mM NaCl, 2.7 mM KCl, 10 mM Na2HPO4, 2 mM KH2PO4, pH 7.4) were transferred into 96-well black microtiter plates (Corning) and fluorescence measured by fluorescence spectroscopy (Spectra MAX GEMINI XS; Molecular Devices) measured with excitations and emission wavelengths of 485 nm and 535 nm, respectively.

### e) Fluoroscence Imaging of Venus YFP PCA Tagged Proteins (Mammalian Cells).

HEK293 cells grown in transparent 24-well plates (DMEM supplemented with 10% FBS) were co-transfected with the Venus YFP PCA expression vectors (pcDNA3.1) coding for prey-F[1] or bait-F[2] using FuGENE6 reagent (Roche). 48 hours after transfection, cells were subjected to fluorescence imaging. Cell images were visualized using a Nikon Eclipse TE2000U inverted microscope with 60× objective and YFP filter cube (41028, Chroma Technologies). Images were captured with a CoolSnap CCD camera (Photometrics) using Metamorph software (Molecular Devices).

### f) In Vitro Protein Binding Assays.

GST fusion proteins (GST, GST-RIIβ or GST-Gαi3) immobilized on glutathione beads were incubated overnight with either RIIβ (Alexis Biochemicals) or with cell lysates from HEK293 cells or HEK293 cells stably expressing the δ-opioid receptor (δOR). Resin-associated complexes were washed at least four times with the lysis buffer (10 mM sodium phosphate pH 7.2, 150 mM NaCl, 0.05% Triton X100 supplemented with standard protease inhibitors) and eluted with Laemmli sample buffer (2% SDS, 50 mM Tris HCl, pH 6.8, 0.2 mg/ml bromphenol blue, 0.1 M DTT, 10% (v/v) Glycerol).

### g) SPOT Synthesis and Overlay Experiments.

Overlapping peptides either of the wild type PKA regulatory subunit RIIβ (accession number: NP_{—}002727) or peptides containing substitutions of conserved amino acids in the Reggai sequence were SPOT-synthesized (Synthetic peptide arrays on membrane supports) on distinct coordinates of a cellulose membrane (shown in FIG. 1c; starting from the N-terminus of RegIIβ with amino acid 1-25 [coordinate A1], amino acid 6-30 [coordinate A2], etc.). Membranes equilibrated in TBST buffer (10 mM Tris, 150 mM NaCl, 0.05% Tween 20, pH 7,4) were overlaid with recombinant GST-Gαi3 (10 μg/ml blocking buffer [TBST supplemented with 5% non-fat dry milk]). Interactions were detected with rabbit anti-GST and secondary horseradish peroxidase antibodies by a procedure identical to immunoblotting (IB), (see Stefan et al., J. Am. Soc. Nephrol. 18, 199-212 (2007)).

h) cAMP Agarose Protein Precipitation Assays.

HEK293, U2OS cells and total brains of mice were homogenized using a Potter S (B. Braun Biotech International) with 15 strikes (lysis buffer: 10 mM sodium phosphate pH 7.2, 150 mM NaCl, 0.05% Triton X100 supplemented with standard protease inhibitors). We clarified the lysate (13000 rpm, 15 min) and precipitated PKA regulatory subunit associated protein complexes with PKA selective 8-AHA-cAMP agarose and Rp-8-AHA-cAMP agarose resin (Biolog, #D014, #M012) for two hours at 4° C. As negative control experiment we added excess of cAMP (5 mM) to the brain lysates to mask the cAMP binding sites in the R subunits for precipitation. Resin associated proteins were washed four times with lysis buffer and eluted with Laemmli sample buffer. Equal amounts of protein (lysates normalized to loading control proteins) were subjected to polyacrylamide gel electrophoresis followed by immunoblotting with indicated antibodies. We performed densitometric quantification of RIIβ, PKAc and Gαi3 (in each case normalized on the most abundant precipitated protein from the same immunoblot (n=3) to compare complex formation and fold enrichment between holoenzyme (precipitated with Rp-8-AHA-cAMP) and activated R complexes (precipitated with 8-AHA-cAMP).

### i) Bioluminescence Resonance Energy Transfer (BRET) Measurement.

δOR-HEK293 cells grown in DMEM supplemented with 10% FBS were transiently transfected with the following constructs: For measurements of the interactions of G protein subunits among each other and with the receptor we transfected Gαi1 with the Renilla luciferase (Rluc) inserted at amino acid position 91 and the UV-shifted GFP variant GFP10 (substitutions: P64L, S147P, and S202P) fused to the C-terminus of Gα2 or the UV-shifted variant GFP2 (substitution: F64L) fused to the C-terminus of the δOR. In overexpression studies, we coexpressed regulatory (RIIβ) or catalytic subunits (PKAc) of PKA. For cAMP-measurements we coexpressed the cAMP sensing Epac in vivo cAMP reporter assay GFP10-Epac-Rluc3 (Rluc3=Rluc variant with following substitutions: A55T, C124A, and M185V) and either RIIβ or PKAc. Forty-eight hours after transfection, cells were treated with combinations of forskolin (100 μM) and SNC80 (1 μM), washed twice with PBS, detached with PBS plus 5 mM EDTA and resuspended in PBS plus 0.1% (w/v) glucose at room temperature. Equal amounts of cells (100,000 cells/well) were distributed in a 96-well microtiter plate (Optiplate, PerkinElmer, Lifescience). Total fluorescence and bioluminescence were evaluated for each well using a FlexStation2 (Molecular Devices) and Mithras LB 940 (Berthold Technologies), respectively. uvGFP fluorescence was excited at 400 nm and read at 515 nm. Fluorescence values were corrected by subtracting the auto-fluorescence of cells not expressing uvGFP. BRET between Rluc and GFP2 or GFP10 was measured after the addition of 5 μM Rluc substrate DeepBlueC (bisdeoxy-coelenterazine, PerkinElmer, Lifescience). BRET signals were derived from the emission detected with the energy acceptor filter (515±15 nm; GFP2/10) divided by the emission detected with the energy donor filter (410±40 nm; Rluc/Rluc3). BRET ratios were corrected by subtracting the background ratio obtained from cells expressing only the energy donor. The conformational rearrangements are either presented as agonist promoted changes of BRET (FIG. 2b), as relative Δ-BRET (FIG. 2c) or as net BRET signals.

### j) MAPK-Phosphorylation Assay (Mammalian Cells).

δOR-HEK293 cells were grown in 12 or 24 well plate formats (DMEM supplemented with 10% FBS). Either 24 or 48 hours after splitting or co-transfection in overexpression studies [transfected with constructs encoding the regulatory (RIIβ) or catalytic subunits (PKAc) of PKA] confluent cells were treated with inhibitors (KT5720 for 60 minutes, pertussis toxin for 16 hours), chemical modulators (forskolin, 6-MBC-cAMP, Sp-5,6-DCI-cBIMPS, Sp-8-PIP-cAMPs, 2-Cl-8-MA-cAMP [all for 15 minutes] or Rp-cAMP for 60 minutes) and/or the δOR ligand SNC80 (for either 3, 6, 10 or 15 minutes). cAMP analogs purchased from Biolog (6-MBC-cAMP, #M012; Sp-5,6-DCI-cBIMPS, #D014; Sp-8-PIP-cAMPs, #P005, 2-Cl-8-MA-cAMP, #C080; Rp-cAMP, #A002S) (see Bobrovskaya et al., Cell. Signal. 19, 1141-1149 (2007); Nguyen et al., Cell. Signal. 16, 1141-1151 (2004); Maronde et al., J. Pineal Res. 27, 170-182 (1999); Ogreid et al., Eur. J. Biochem. 181, 19-31 (1989); Poppe et al., Nat. Methods 5, 277-278 (2008)).

Following treatments at 37° C. for 3, 6, 10 or 15 minutes the reactions were stopped by direct addition of Laemmli sample buffer. Equal amounts of protein (lysates normalized to loading control proteins) were subjected to polyacrylamide gel electrophoresis followed by immunoblotting (IB). To analyze the phosphorylation of Erk1/2 we applied the Phospho-p44/42 MAP Kinase antibody (Cell Signaling Technology, #9101). Quantitative densitometry of immuno blot bands was performed using the program ImageQuant 5.2 (Molecular Dynamics).

### k) Renilla Luciferase PCA.

We have generated a stable HEK293T cell line coexpressing the PCA fusion proteins RIIβ-Rluc-F[1] and PKAc-RlucF[2], as previously described. All cells were grown in DMEM supplemented with 10% FBS. We treated confluent HEK293T cells (stably expressing the PKA reporter) grown in 24 well plate format with varying concentrations and combinations of cAMP analogs 6-MBC-cAMP and Sp-5,6-DCI-cBIMPS (15 min; Biolog, Germany). We pretreated confluent HEK293T cells (stably expressing the PKA reporter) grown in 24 well plate format with 250 μM of Rp-cAMP (60 minutes) or with 6-MBC-cAMP and Sp-5,6-DCI-cBIMPS (=RII selective, each 50 μM; 15 min). We pretreated confluent HEK293T cells (stably expressing the PKA reporter) grown in 24 well plate format for 30 minutes with TAT or Reggai fusion peptides (˜3 μM) followed by exposure to forskolin (100 μM; 15 min). Following treatment, growth medium was exchanged and cells were resuspended in 350 μl of PBS. 100 μl of cell suspensions were transferred to 96-well plates and subjected to bioluminescence analysis using the LMaxTMII384 luminometer (Molecular Devices). Rluc bioluminescence signals were integrated for 10 seconds following addition of the Rluc substrate benzyl-coelenterazine (5 μM; Nanolight). Decrease of bioluminescence signals indicates dissociation of the RIIβ-RlucF[1]:PKAc-RlucF[2] complex along with activation of the kinase activity of the PKA catalytic subunit (see Stefan et al., Proc. Natl. Acad. Sci. U.S.A. 104, 16916-16921 (2007)).

### Example 1

### Direct Protein: Protein Interaction Between PKA RIIβ Subunits and Gαi

In mammalian COS7 cells, we identified novel interactions of PKA RIIβ/Reg subunits, but not PKAc/Cat subunits with all three isoforms of Gαi (Gαi1,2,3) using a ‘Venus’ yellow fluorescent protein (YFP) protein-fragment complementation assay (PCA) (FIG. 1b);

In contrast to the Gαi isoforms, we observed another novel interaction between the Phosphoinositide-dependent kinase 1 (PDK1) and both PKA RIIβ and Catα subunits, suggesting different modes of binding for this protein. RIIβ:RIIβ and PDK1:RIIβ complexes were localized to the cytosol, but the Gαi:RIIβ complexes were localized to the plasma membrane, consistent with known localization of Gαi proteins (FIG. 1b). We confirmed the RIIβ:Gαi3 interaction in glutathione S-transferase (GST) pulldown assays. GST-RIIβ precipitates Gαi3 and GST-Gαi3 precipitates purified RIIβ.

In dot blot screens of overlapping peptides derived from RegIIβ, we identified a unique binding motif (SEQ ID No. 1 to 8) for Gαi3 within the evolutionarily conserved cyclic nucleotide binding domain B (CNB-B) (FIG. 1c, d; see FIG. 4).

FIG. 4 clearly shows that the presence of the core sequence of the Reggai peptide, in particular the Reggai peptide, is essential to allow binding to Gαi.

We then tested whether pre-dissociating endogenous R/Reg and PKAc/Cat subunits would change the stoichiometry of R:Gαi3 or R:PDK1 complexes. Proteins named without suffixes refer to endogenous proteins of undetermined isoform (e.g. R/Reg, PKAc/Cat) and those named with suffixes refer to specific isoforms transiently overexpressed in cells (Gαi3, Pdk1). We treated COS7 cells with forskolin, a general activator of AC activity, for 15 minutes, lysed and applied lysates to 2AHA-cAMP-coupled agarose beads. Under basal conditions endogenous Reg subunits bound to 2AHA-cAMP-coupled agarose beads coprecipitated overexpressed Gαi3 and PDK1.

There was no difference in the stoichiometry of R:PDK1 complexes. However, pre-treatment of cells with forskolin caused a significant, 3.8 (SD±0.99) fold enrichment of R:Gαi3 complexes. This result is remarkable because to date, the only functions attributed to Reg subunits is to inhibit catalytic PKA activity through binding to PKAc/Cat subunits and to mediate the constitutive interaction with A kinase anchoring proteins (AKAP) which target the holoenzyme to its substrates.

These results suggest that cAMP binding to R mediates dissociation of the R₂:PKAc₂ complex, but may also mediate the active association of R/Reg subunits to Gαi proteins and perhaps modulate their activity.

### Example 2

### Augmentation of Gαi-Activity by R:Gαi Complex

To examine the potential role of R:Gαi complexes in GPCR signalling, we used HEK293 cell line stably expressing the δ-opioid receptor (δOR), a prototypical Gαi-coupled GPCR (see Pineyro et al., Molecular pharmacology 60, 816-827 (2001)). First we determined how formation of cAMP-triggered RII:Gαi complexes could effect δOR coupling to and activation of Gαi. For this purpose, we utilized two bioluminescence resonance energy transfer (BRET)-based assays. The first assay reports on a known conformational rearrangement of the trimeric Gαi:βγ complex that occurs upon activation of Gαi (see Gales et al., Nat Struct Mol Biol 13, 778-786 (2006)). The second assay reports on a known change in Gαi1 coupling to δOR in response to agonist binding to the GPCR (see Gales et al., Nat Struct Mol Biol 13, 778-786 (2006)). We observed that the δOR agonist SNC80 induced a decrease in BRET between Gαi1 and Gγ2, reflecting activation of the G-protein. Interestingly, direct activation of cAMP production by forskolin further potentiated the Gαi1:Gγ2 conformational rearrangement resulting from receptor activation, suggesting that cAMP-mediated activation of PKA causes enhanced activation of Gαi, possibly via the formation of the R:Gαi1 complex. Forskolin treatment alone had no effect on the conformation of the trimeric G-protein complex.

Further, overexpression of RIIβ, but not of catalytically active PKAc/Cat, enhanced both SNC80 and SNC80+forskolin effects (FIG. 2a). Also consistent with the teaching that the formation of the R:Gαi1 complex potentiates the receptor-mediated activation of Gαi1, we observed that forskolin potentiated the SNC80-induced increase in BRET between δOR and Gαi1, an indication of increased engagement of Gαi by the receptor.

### Example 3

### RI/RegI versus RII/RegII

We next tested whether the activation of distinct PKA R/Reg isoforms (RegI or RegII) affect signalling through Gαi3. Therefore we directly activated the PKA holoenzyme with combinations of cAMP analogues that bind selectively either to CNB-A or CNB-B of RI/RegI or RII/RegII, thereby inducing the dissociation of PKAc/Cat subunits. In contrast to RegI subunit activators (8-PIP-cAMP, 2-Cl-8-MA-cAMP) that had no effect, the RI/RegI activators (6-MBC-cAMP, Sp-5,6-DCI-cBIMPS) potentiated the amplitude and duration of SNC80-induced Erk1/2 phosphorylation (FIG. 3a).

A dose-response of SNC80-mediated ERK1/2 phosphorylation in the presence and absence of RII/RegII activators showed a seven-fold increase in sensitivity of the MAPK pathway to stimulation by the δOR agonist (FIG. 3b).

Our teaching predicts that preventing cAMP-triggered PKA dissociation and hence formation of RII:Gαi complexes would prevent Gαi-coupled receptor signal augmentation by cAMP. To support this, we used the cAMP analogue Rp-cAMP, an inhibitor of PKA holoenzyme dissociation. Pre-treatment with Rp-cAMP decreased SNC80+RegII subunit activator (6-MBC-cAMP, Sp-5,6-DCI-cBIMPS) and SNC80+forskolin mediated Erk1/2 phosphorylation to a similar extent as it prevented PKA holoenzyme dissociation (FIG. 3d).

Finally, we showed that overexpression of RIIβ/RegII [symbol] but not catalytically active PKAc/Catα further enhanced the potentiation of SNC80+RII/RegII subunit activating cAMP analogues. Transient overexpression of RIIβ/RegIIβ subunits had no detectable impact on the activation of δOR with SNC80 alone.

This data further supports our teaching that conformational changes in RII/RegII subunits upon cAMP binding allows for both PKAc/Cat release and activation, and binding to and augmented signalling through receptor-activated Gαi.

### Example 4

### R:Gαi Complex Augmented Gαi-Activity Reduces cAMP Synthesis and Augmented Signal Transmission to MAPK

Since activated Gαi is known to inhibit AC, we predicted that cAMP-bound RIIβ/RegIIβ-mediated augmentation of Gαi activity should itself reduce cAMP synthesis. We tested this hypothesis using a BRET cAMP reporter based on the structure of the cAMP-regulated guanine nucleotide exchange factor (EPAC) that undergoes conformational changes upon binding to cAMP. This assay provides for accurate detection of in vivo cAMP levels indicated through a decrease of BRET (see Leduc et al., J Pharmacol Exp Ther 331, 297-307 (2009)). We observed that overexpression of RII [symbol] but not of Catα, resulted in a further decrease in cAMP production when cells were treated with SNC80+forskolin compared to cells treated with forskolin alone (FIG. 2b).

We also tested the impact of R:Gαi complex formation on the evolutionary conserved route of signal transmission from Gαi to MAPK9. SNC80 induced a large but transient increase in Erk1/2 phosphorylation in δOR-HEK293 cells. Pre-treatment with forskolin potentiated the SNC80-induced Erk1/2 phosphorylation (FIG. 2c). This data supports the notion that the cAMP-dependent formation of the R:Gαi complex also potentiated this branch of the Gαi signalling activity. To exclude the possibility that PKA kinase activity could account for this potentiation, we pre-treated cells with the PKA inhibitor KT5720 (unless noted otherwise, all subsequent experiments were carried out in the presence of this kinase activity inhibitor). We confirmed that KT5720 inhibits PKA kinase activity under the conditions used by monitoring CREB phosphorylation (activated independently of Gαi signalling).

### Example 5

### RIIβ/RegIIβ-Derived Peptide Called Reggai

We created a membrane-permeable peptide that competes for the Gαi:R interaction based on a hybrid peptide composed of a fusion between the membrane-permeating HIV TAT and 15 amino acids derived from the only Gαi3 binding motif that we identified in the RIIβ/RegIIβ subunit referred to here as Reggai (FIG. 1c,d). Treatment of cells with the Reggai peptide prevented the potentiation of SNC80-mediated Erk1/2 phosphorylation using RII/RegII subunit activators (FIG. 3e). A TAT control peptide had no effect. Neither Reggai nor TAT control peptide pre-treatment of HEK293T cells had any effect on PKA holoenzyme dissociation.

1. A PKA RII-derived peptide consisting of 12 to 17 amino acids, wherein the PKA RII-derived peptide comprises the amino acid sequence DSFXIXE (SEQ ID No. 1) or XXXXXDSFXIXEXXX (SEQ ID No. 4), and wherein X is any amino acid.

2. The peptide according to claim 1, comprising the amino acid sequence DSFFIVE (SEQ ID No. 2) or XXXXXDSFFIVEXXX (SEQ ID No. 5), wherein X is any amino acid.

3. The peptide according to claim 1, comprising the amino acid sequence DSFYIIE (SEQ ID No. 3) or XXXXXDSFYIIEXXX (SEQ ID No. 6), wherein X is any amino acid.

4. The peptide according to claim 1, which is QGDSADSFFIVESGE (SEQ ID No. 7) or QGASADSFFIVESGE (SEQ ID No. 8).

5. The peptide according to claim 1, which is fused to another peptide or protein.

6. The peptide according to claim 1, which is labelled.

7. (canceled)

8. A method for treating cancer in a subject in need thereof, the method comprising administering to the subject the peptide according to claim 1.

9. A method for treating a hormone-related disease or heart failure in a subject in need thereof, the method comprising administering to the subject the peptide according to claim 1

10. (canceled)

11. A pharmaceutical composition comprising the peptide according to claim 1 and a pharmaceutically acceptable carrier.

12. The pharmaceutical composition of claim 11, comprising a further pharmaceutically active agent.

13. A method for screening candidate drugs, the method comprising:
a) providing a peptide structure comprising peptide according to claim 1 and at least one candidate drug,
b) bringing the compounds of step a) in physical contact with each other under binding conditions and
c) analysing the binding of the at least one candidate drug to the peptide structure.

14. The method of claim 13, wherein in step a) a cAMP-bound PKA RII subunit, a G protein αi or both are provided and brought into physical contact with the other compounds in step b).

15. A method for screening candidate drugs, the method comprising:
d) providing a cAMP-bound PKA RII subunit, a G protein αi (Gαi) and at least one candidate drug,
e) bringing the compounds of step a) into physical contact with each other under binding conditions and
f) analysing the effect of the candidate drug on the binding of the at least one cAMP-bound PKA RII subunit to Gαi.

16. A method of preparing cAMP-bound PKA RII subunit, the method comprising:
g) providing a source containing cAMP-bound PKA RII subunit and a source containing G protein αi,
h) contacting the source containing cAMP-bound PKA RII subunit with the source containing G protein αi under conditions allowing for the binding of cAMP-bound PKA RII subunit to Gαi,
i) obtaining the bound complex of PKA RII subunit/Gαi and
j) separating the PKA RII subunit from Gαi.

17. A method of preparing G protein αi comprising
k) providing a source containing cAMP-bound PKA RII subunit or a peptide according to claim 1 and a source containing Gαi,
l) contacting the source containing cAMP-bound PKA RII subunit or the peptide according to claim 1 with the source containing Gαi under conditions allowing for the binding of cAMP-bound PKA RII subunit or the peptide according to claim 1 to Gαi,
m) obtaining the bound complex of PKA RII subunit/Gαi or of the peptide according to claim 1/Gαi and
n) separating Gαi from the PKA RII subunit or the peptide according to claim 1.

18. The method according to claim 13, wherein PKA RII is PKA RIIβ.

19. The method according to claim 15, wherein PKA RII is PKA RIIβ.

20. The method according to claim 16, wherein PKA RII is PKA RIIβ.

21. The method according to claim 17, wherein PKA RII is PKA RIIβ.
